(19) United States
(12) Patent Application Publication
(10) Pub. No.: US 2017/0122616 A1
(43) Pub. Date: May 4, 2017

(54) SMART MULTI-FUNCTIONAL CEILING UNIT

(71) Applicant: Marley Engineered Products LLC, Michigan City, IN (US)

(72) Inventor: Jeffrey Calabro, Michigan City, IN (US)

(21) Appl. No.: 14/929,783

(22) Filed: Nov. 2, 2015

(51) Int. Cl. F24F 11/00; G10L 15/22; A61L 9/015; H04R 1/02; H05B 37/02; F24F 7/007

(52) U.S. Cl. CPC F24F 11/0086; H05B 37/0218; F24F 11/0034; F24F 11/0015; F24F 11/0079; F24F 7/007; A61L 9/015; H04R 1/028; G10L 15/22; F24F 2221/02; F24F 2221/14; F24F 2011/0068; F24F 2011/0057; F24F 2013/247

(57) ABSTRACT

A ventilation fan and light system is provided. The system includes: a controller; a sensor operatively connected to the controller; a fan switch operatively connected to the fan; a light operatively connected to the controller; a dimmer operatively connected to the light and the controller to operate the light at various levels of brightness; a light switch operatively connected to the controller and configured to selectively operate the light; and a first input device operatively connected to the controller to input to the controller a signal associated with any one of the following: a time of day and a detected level of ambient light.

### FIELD OF THE INVENTION

The present disclosure relates generally to ventilation systems. More particularly, the present disclosure relates to a ventilation system that provides additional functions in addition to ventilation.

### BACKGROUND OF THE DISCLOSURE

Domestic ventilation systems such as bathroom fans typically provide a fan for ventilating the bathroom and, in some instances, include a light. Additional bathroom fan systems may also include a heater such as an infrared heater for providing heat for the bathroom. Other ventilation systems may be made more robust for commercial application that often provide similar function as those described above with respect to domestic ventilation systems.

As home appliances and systems are becoming more and more sophisticated, more functions may be available from home appliances. As a result, new bathroom fan or ventilation systems may additional functions than typically found.

Accordingly, it is desirable to provide a method and apparatus that provides additional capabilities then typical systems that include only ventilation, light, and heat.

### SUMMARY OF THE INVENTION

The foregoing needs are met, to a great extent, by the present disclosure, wherein in one aspect an apparatus is provided that in some embodiments a ventilation system is provided that can accomplish many functions.

In accordance with one embodiment of the present disclosure, a ventilation fan and light system is provided. The system includes: a controller; a sensor operatively connected to the controller; a fan switch operatively connected to the fan; a light operatively connected to the controller; a dimmer operatively connected to the light and the controller to operate the light at various levels of brightness; a light switch operatively connected to the controller and configured to selectively operate the light; and a first input device operatively connected to the controller to input to the controller a signal associated with any one of the following: a time of day and a detected level of ambient light.

In accordance with another embodiment of the present disclosure, a method of providing ventilation and light is provided. The method includes: connecting a light to a controller; connecting a fan to the controller; connecting a dimmer to the light and the controller; connecting a switch to the controller; and configuring a signal generator to generate a signal to the controller wherein the signal is associated with one of either a time of day and an amount of ambient light.

In accordance with yet another embodiment of the present disclosure, a ventilation fan and light system is provided. The system includes: a means for controlling fan and light system; a means for sensing connected to the means for controlling; a means for switching operatively connected to a means for moving air; a light operatively connected to the means for controlling; a means for controlling brightness of the light operatively connected to the means for controlling; a means for switching operatively connected to the light; and a means for inputting data to the means for controlling, the means for inputting data configured to input data associated with one of either: a time of day and an amount of light detected by the means for sensing.

There has thus been outlined, rather broadly, certain embodiments of the disclosure in order that the detailed description thereof herein may be better understood, and in order that the present contribution to the art may be better appreciated. There are, of course, additional embodiments of the disclosure that will be described below and which will form the subject matter of the claims appended hereto.

In this respect, before explaining at least one embodiment of the disclosure in detail, it is to be understood that the disclosure is not limited in its application to the details of construction and to the arrangements of the components set forth in the following description or illustrated in the drawings. The disclosure is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

### BRIEF DESCRIPTION OF THE DRAWINGS

FIG. 1 is a perspective view illustrating a ceiling unit according to the disclosure.

FIG. 2 is a bottom view of a ceiling unit according to the disclosure.

FIG. 3 is a side view of a wall-mounted remote control according to the disclosure.

FIG. 4 is a schematic diagram of a smart multifunctional unit according to the disclosure.

FIG. 5 is a side view of an air freshener dispenser associated with the smart multifunctional unit according to the disclosure.

### DETAILED DESCRIPTION

The disclosure will now be described with reference to the drawing figures, in which like reference numerals refer to like parts throughout. An embodiment in accordance with the present disclosure provides a smart ventilation system that has a variety of features. For example, in some embodiments, the ventilation system may include some or all of the following features: lighting, fan control for ventilation and odor control, humidity control, infrared heat, provide audio, noise cancellation, air freshening, and voice and/or remote control.

In many applications the ventilation system may be a ceiling fan unit for use in a room such as a restroom or bathroom. Many residential bathroom fan or ventilation units are commonly located in the ceiling. For this reason, ventilation units are often referred to as ceiling units. While the present disclosure will refer to a ceiling unit or ceiling fan unit it should be understood that the actual location of the unit is not necessarily limited to a ceiling. Rather the unit may be located in any place that will render a desired effect. Wall-mounted, floor mounted, freestanding or any other suitable location for the unit is contemplated and may be encompassed by the appended claims.

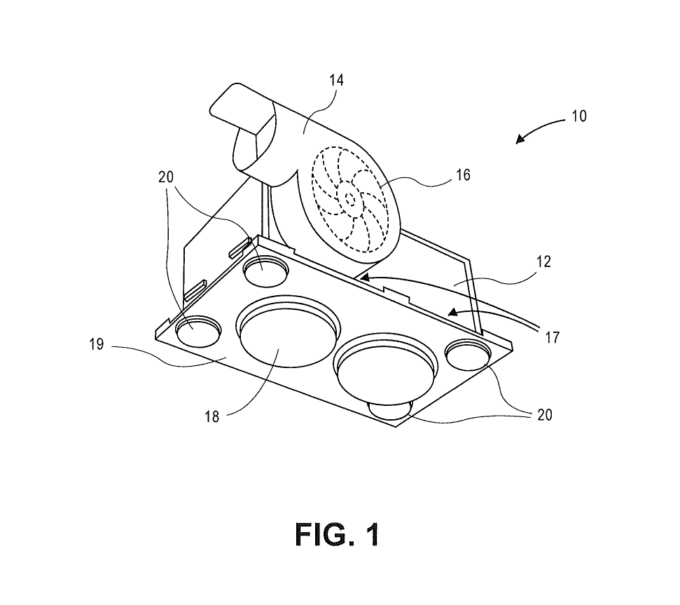

An embodiment of the present inventive apparatus is illustrated in FIG. 1. FIG. 1 illustrates a ventilation unit 10 having a housing 12. The ventilation unit 10 may also include a discharge housing 14 which, in some embodiments, may house a fan 16 shown in phantom lines as it is located within the discharge housing 14. In other embodiments, a fan 16 or other air moving device may be located within the housing 12. As such, the discharge housing 14 is an optional feature. The fan 16 may be a radial fan, squirrel cage type fan, axial fan, or any other type of fan may also be used. Furthermore, it should be understood that the ventilation unit 10 is not limited solely to having a fan but any other mechanism for moving air and providing ventilation may be used in accordance with the present disclosure.

The ceiling unit 10 may also include openings 17 which allow air to enter the housing 12 to be processed by the fan 16 and discharged. One of ordinary skill the art after reviewing this disclosure will understand that a discharge hose or conduit (not shown) may be attached to the discharge housing 14 in any suitable manner to discharge the air to the outside or other suitable place of deposit. Because such discharge connections are well known, they will not be described further herein. Other embodiments may locate the openings 17 through the housing 12 or in other locations. For example, the cover plate 19 may have several openings and appear as a grill. Other locations for the openings 17 may also be used in accordance with the disclosure.

The ceiling unit 10 may include heat lamps 18. In some embodiments, the heat lamps 18 are simply infrared light bulbs or any other type of infrared heat source operatively attached to the ventilation unit 10. Attaching the heat lamps 18 may be accomplished by screwing the heat lamps 18 into light sockets or any other suitable manner of operatively connecting infrared lamps 18 to a ventilation unit 10. The infrared lamps 18 may be configured to provide heat at areas within a line of sight from the infrared lamps 18. In other embodiments, other types of heaters such as, but not limited to, resistance heaters or any other suitable device for providing heat may be used and attached to the ventilation unit 10 in a manner similar described with respect to the infrared lamps 18.

In some embodiments, the ventilation unit 10 will include lights 20. The lights 20 can be incandescent lights, fluorescent lights, LED lights, or any other suitable type of lights.

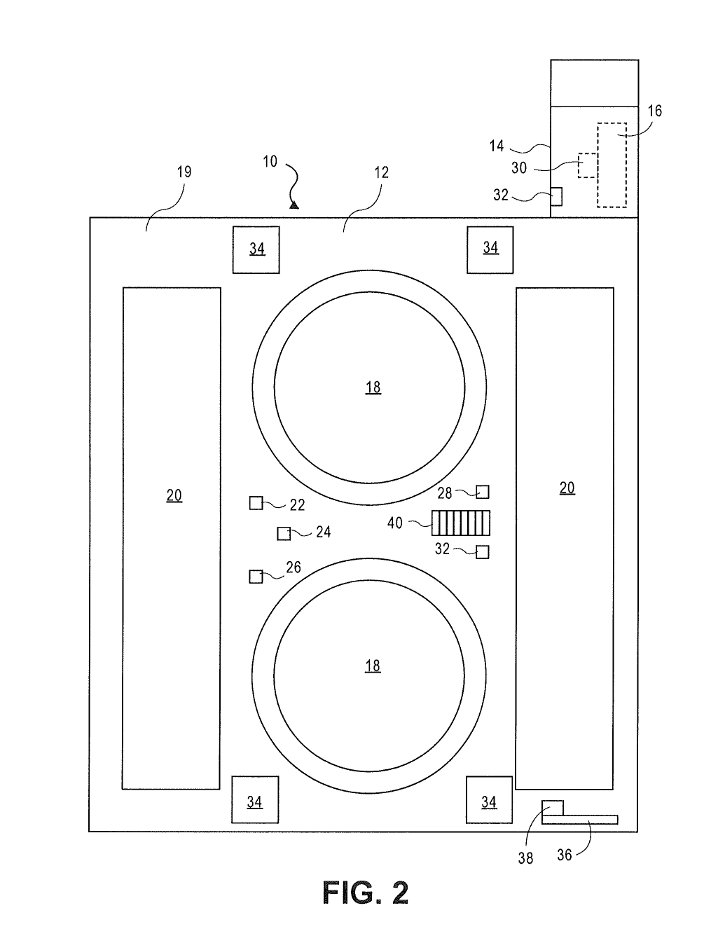

FIG. 2 illustrates a bottom view of a ventilation unit 10 in accordance with another embodiment. The ventilation unit 10 of FIG. 2 includes a housing 12. The housing 12 also includes a discharge housing 14 similar to that described above with respect to FIG. 1. A fan 16 operated by a motor 30 (shown in phantom lines as the motor 30 and fan 16 are inside the discharge housing 14). The ventilation unit 10 of FIG. 2 includes infrared lamps 18 for providing heat and lights 20 providing light. The infrared lamps 18 and the lights 20 may extend through the cover plates 19. The ventilation unit 10 may include a motion sensor 22, a light sensor 24, odor sensor 26, and a humidity sensor 28.

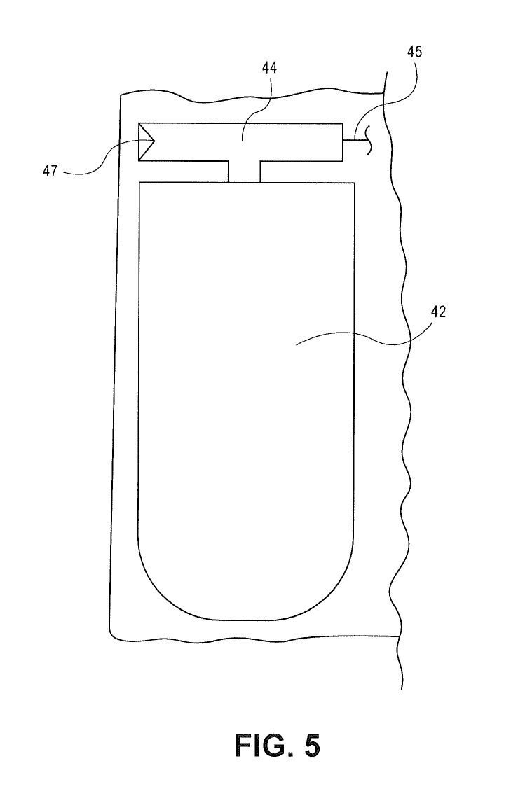

As shown in FIG. 2, the ventilation unit 10 may include a microphone 32 and speakers 34. An antenna 36 for a wireless link 38 is also shown. The wireless link 38 may be a Wi-Fi connection, Bluetooth, radio, infrared or any other suitable wireless connection. In some embodiments, the ventilation unit 10 will include an air freshener grill 40 behind which an air freshener reservoir 42 and air freshener actuator 44 (the reservoir 42 and actuator 44 shown and described in additional detail later below respect to FIG. 5) may be located.

It should be understood that the various features such as the shape, location, or placement of the housing 12, the shape and even whether there is a discharge housing 14, a fan 16, openings 17, infrared lamps 18, cover plates 19, lights 20, a motion sensor 22, a light sensor 24, odor sensor 26, a humidity sensor 28, a motor 30, a microphone 32, speakers 34, an antenna 36 for the wireless link 38, an air freshener grill 40, an air freshener reservoir 42, (see FIG. 5) an air freshener dispenser 44 (see FIG. 5) can vary in different embodiments. Various ventilation units 10 in accordance with the present disclosure may have some, all, and perhaps even other features than those described herein. Furthermore, the layout and configurations meant to be exemplary and not limiting.

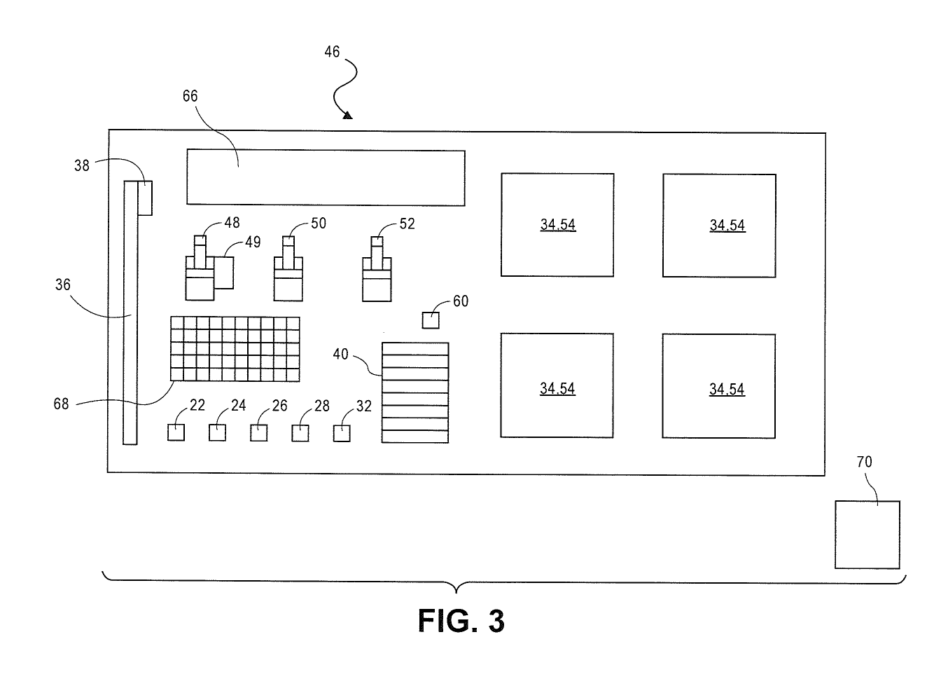

In some embodiments, some of the features may not be located on the ventilation unit 10 itself but rather in a remote device 46 as shown in FIG. 3. For example, a remote device 46 may be a wall mounted unit located proximate to or similar to light switches on a wall.

The remote device 46 illustrated in FIG. 3 is an example of a remote device 46 that may be wall mounted. The remote device 46 includes a light switch 48, fan switch 50, and a heat switch 52. The switches 48, 50, and 52 may be on-off switch, similar to normal light switches. In other embodiments, the switches 48, 50, and 52 may have detents which allow the switches to be in specific settings between full on and full off, or in still other embodiments, the switches 48, 50, and 52 may be able to be adjusted to an infinite amount positions to adjust the lights fan or here is to various degrees of intensity.

In embodiments where the speakers 34 are located on the ventilation unit 10, the remote unit 46 may include additional speakers 54. In embodiments where no speakers are located on the ventilation unit 10, then the first set of speakers 34 may be located on the remote unit 46. In some embodiments, features described above to be located on the ventilation unit 10 may be instead or, in addition to, located on the remote unit 46. For example in some embodiments, the remote unit 46 may also include motion sensor 22, a light sensor 24, and odor sensor 26, a humidity sensor 28, and a microphone 32. In some embodiments, the remote unit 46 may also include an air freshener grill 40 behind which an air freshener reservoir 42 and air freshener actuator 44 (shown and described in more detail below with respect to FIG. 5) may be located.

The remote unit 46 may also include an antenna 36 operatively connected to a wireless link 38 for wirelessly connecting to the ventilation unit 10. In other embodiments, the remote unit 46 may communicate with the ventilation unit 10 with a wire or wires in a conventional manner. In some embodiments, the remote unit 46 may also include an additional sensor 60 to provide other input. In some embodiments, the remote unit 46 may include a user display 66. The user display 66 may be a screen which can indicate various settings or messages to a user. In some embodiments, a user input 68 may be in the form of buttons or a keypad that is located on the remote unit 46. The user display 66 and the user input device 68 may converge to a single apparatus such as a touchscreen.

In some embodiments, a second remote unit 70 may be used in addition to the first remote unit 46. In such instances, the second remote unit 70 may be a remote control, a smart phone, a computer, tablet device, or any other similar remote device. The second remote device 70 may communicate with the first remote device 46 and/or the ventilation unit 10.

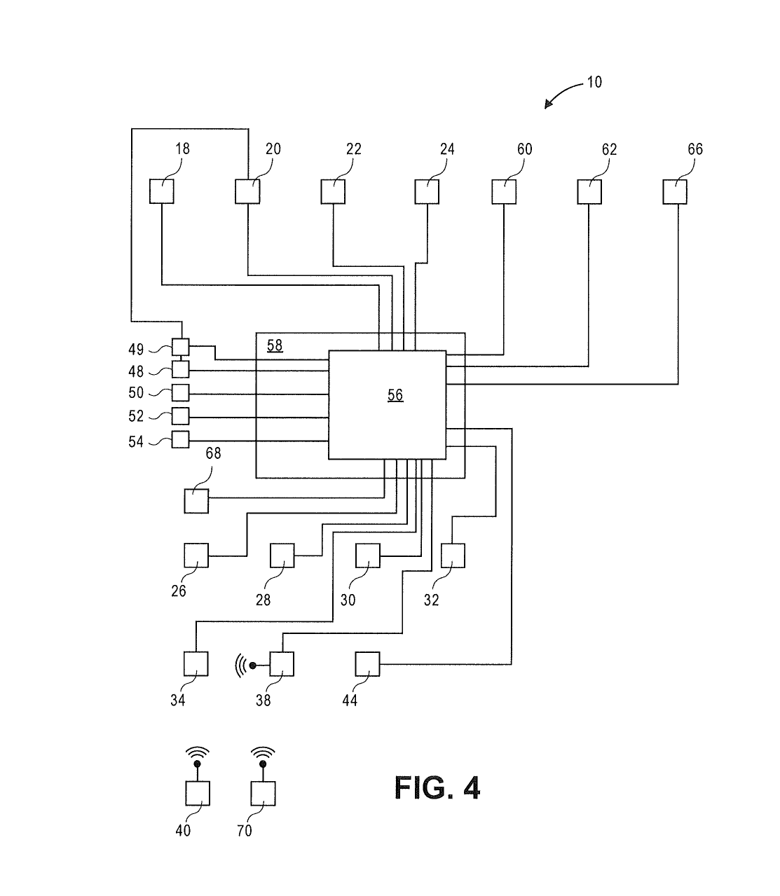

FIG. 4 is a schematic view of the ventilation unit 10 that may be controlled by a controller 56. The controller 56 may be a microcontroller 56 mounted to a PCB board 58. The microcontroller 56 may have various inputs and outputs in order to operate the ventilation unit 10. For example, the microcontroller 56 may be operatively connected to the motor 30 which operates the fan 16 (shown in FIGS. 1 and 2). The microcontroller 56 may also be operatively connected to the infrared lamps 18 as well as the lights 20. The microcontroller 56 may have a built-in dimmer or dimmer function programmed in the microcontroller 56 in order to dim the lights 20 to a desired level. In other embodiments, the light switches 48 themselves may be operatively connected to a dimmer 49 to dim the lights 20 (see FIG. 2). The microcontroller 56 can also operate the infrared lamps 18 to put out a desired level of heat.

The motion sensor 22 is operatively connected to the microcontroller 56 to input a signal to the microcontroller 56 associated with a detected motion. The light sensor 24 is operatively connected to the microcontroller 56 and configured to input a signal to the microcontroller 56 regarding an intensity of ambient light that is measured or detected by the light sensor 24.

An odor sensor 26 is operatively connected to the microcontroller 56 to input a signal associated with a detected odor to the microcontroller 56.

A humidity sensor 28 is operatively connected to the microcontroller 56 and configured to input a signal to the microcontroller 56 indicative of an amount of humidity sensed or detected by the humidity sensor 28. A microphone 32 is operatively connected to the microcontroller 56 and configured to input a signal to the microcontroller 56 regarding sound is detected by the microphone 32. Speakers 34 are operatively connected to the microcontroller 56 and configured to operate as controlled by the microcontroller 56.

A wireless link 38 is operatively connected to the microcontroller 56 in order to allow signals inputted from remote devices 46 and 70 to be inputted into the microcontroller 56. In addition, the microcontroller 56 can send signals via the wireless link 38 to the remote units 46 and 70.

An air freshener actuator 44 may be operatively connected to the microcontroller 56 in order to allow the microcontroller 56 to control dispensing of an air freshener from the air freshener reservoir 42 (see FIG. 5). The light switch 48, the fan switch 50, and the heat switch 52 may be operatively connected to the microcontroller 56 so that settings of the light switch 48, fan switch 50, and the heat switch 52 set by a user can be input into the microcontroller 56.

Optionally, the second set of speakers 54 may also be operatively connected to the microcontroller 56 so the microcontroller 56 can control the second set of speakers 54. Other sensors 60 may also be operatively connected to the microcontroller 56 so that additional sensed parameters can be inputted into the microcontroller 56. Additional outputs for actuators may also be operatively connected to the microcontroller 56 so that the microcontroller 56 can operate other actuators or outputs 62. The user display 66 and the user input device 68 are also operatively connected to the microcontroller 56 so messages from the microcontroller 56 can be displayed to the user and commands may be input to the microcontroller 56 by a user.

While many of the sensors and/or actuators are shown to be directly connected to the microcontroller 56, it should be understood that many of the actuators and/or sensors may be located on or at the first 46 and/or second remote device 70. As such, the sensors/actuators may be operatively connected to the microcontroller 56 wirelessly or by some other link. For example, the sensors and/or actuators may communicate to the microcontroller 56 via the wireless link 38 located on the first 46 and/or second remote device 70.

FIG. 5 illustrates an air freshener reservoir 42 which is configured to store an amount of air freshener. In some instances the air freshener reservoir 42 may be a replaceable cartridge. In other instances the air freshener reservoir 42 may be refilled by a user. An air freshener actuator 44 is configured to dispense air freshener from the air freshener reservoir 42 into the bathroom or whatever room the ventilation unit 10 is located. The air freshener actuator 44 may pump air freshener out of the reservoir 42. In other embodiments, the actuator 44 may open a valve to release pressurized air freshener from the reservoir 42. In other embodiments, the actuator 44 may release air freshener using any other suitable means. The freshener actuator 44 can be connected via a communication link 45 to the microcontroller 56. In some instances the communication link 45 may be wired or in other instances it may be a wireless connection 38.

Various embodiments of the disclosure may include ventilation units 10 having different combinations of features. For example, some ventilation units 10 may optionally include the infrared lamps 18. Some may optionally include motion sensors 22. Speakers 34 and the air freshener reservoir 42 are also optional features. It should be understood that one of ordinary skill the art, after reviewing this disclosure, will understand various combinations that may be included. As result, and due the fact there are so many different variations possible, no attempt to categorize and describe each variation individually will be done herein.

In some embodiments, the fan 16 may be operated by a fan switch 50 located on a remote unit 46. In some embodiments the remote unit 46 is mounted on a wall in the bathroom or same room as the ventilation unit 10 in other embodiments, the remote unit 46 may be used in addition to the second remote unit 70. The second remote 70 unit may be a smart phone, a tablet computer, personal computer or any other remote device. The second remote unit 70 may send control signals to the microcontroller 56 via the first remote unit 46. Optionally, the second remote unit 70 may bypass the first remote unit 46 and send control signals to the microcontroller 56 via the wireless link 38. Signals from the second remote unit 70 may also be transmitted by wires. Some embodiments may not utilize the first wall mounted remote device 46 and only use a second remote device 70.

In embodiments having a humidity sensor 28, the fan 16 may also be operated by the microcontroller 56 when a humidity level is detected above a certain threshold. The threshold may be set at the factory or may be programmed by a user via the user input device 68. As result, the fan 16 may be operated by the fan switch 50 or automatically operated within a humidity level is exceeded.

In some embodiments, the ventilation unit 10 is equipped with a motion sensor 22. The motion sensor 22 may be configured to detect motion within the room in which the ventilation unit 10 is mounted. For example, if a user enters the room in which the ventilation unit 10 is located, a motion sensor 22 may detect that motion and operate the lights 20 to turn on the lights 20 via the microcontroller 56.

The microcontroller 56 may be programmed to initially operate the lights 20 at a dimmed level depending upon the time of day or, if there is a light sensor 24, if ambient light is sensed. For example if, in the middle of the night, a user walks into the restroom in which the ventilation unit 10 is located, the lights may initially be turned dim and then gradually brighten as to not make the user be uncomfortable due exposure to the full brightness of the light all at once. The microcontroller 56 may have a dimmer routine programmed into it to allow the lights to gradually be brightened. In some embodiments, the percentage of light initially turned on in response to a signal from the motion sensor 22 may be programmed into the microcontroller 56 or be set by a user via a user input device 68.

For example: If someone enters the bathroom at 2:00 AM, the motion sensor will detect this person and gradually turn on the lights from 0% to 20% instead of traditional lights turning on at 100% which can cause the user eye pain. If someone entered the bathroom at 4:00 PM, the unit would turn on the lights at 100%. If the motion sensor does not detect movement after a user selected or factory program amount of time, the lights will shut off automatically.

The light switch 48 may also cause the lights 20, to be activated in a graduated manner or can be an override to cause the lights to come on at full brightness instantly depending upon the settings set either the factory or by user via the user input device 68.

In some embodiments, the microcontroller 56 will detect one or both of a time of day and an ambient light condition as sensed by a light sensor 24 to determine how bright to turn on the lights 20. For example, during typical working hours such as 6 AM to 10:00 PM the microcontroller 56 may always turn the lights on a full brightness however during typical sleeping hours 10:01 PM to 5:59 AM the lights may initially be dimmed and then gradually go brighter. The times for dimming the lights are examples only and may be altered or programmed by a user as desired. In alternative embodiments, the microcontroller 56 will control the lights 20 to be initially dimmed if not much light is sensed by a light sensor 24, but, if the light sensor 24 senses a lot of ambient light, then the lights 20 will be programmed to turn on brightly. The exact parameters may be modified by a user, or in some instances, preprogrammed by the manufacture for individual applications and individual desires.

Some embodiments may be equipped with an odor sensor 26. The odor sensor 26 is operatively connected to the microcontroller 56. When equipped with an odor sensor 26, the microcontroller 56 will operate an air freshener actuator 44 in order to spray an air freshener into the room in which the ventilation unit 10 is located if the detected odor sensed by the odor sensor 26 exceeds a certain threshold. This threshold can be set at the factory during manufacturing or the modified by a user via the user input device 68. In some embodiments, the fan 16 may also be operated by the microcontroller 56 in response to odor detected by the odor sensor 26. In such cases, if an odor is detected to exceed a certain threshold, the fan 16 may be operated until the intensity of the odor detected goes below the threshold.

In some embodiments, the odor sensor 26 will be used to “learn” what the normal scent of the room is. The user can also calibrate the sensor by giving the voice command, “Learn Scent” or by using the user input device 68. If the odor sensor 26 detects the room's scent is outside of a “learned” scent, the unit will automatically disperse a small spray of air freshener into the room. The user can also use the voice command, “Fresh” to activate the air freshener. The user can also activate the air freshener via the unit's wall control remote unit 46 or second remote device 70. The air freshener replacement cartridge level is monitored by the unit, so when the freshener is almost out, the unit can automatically order replacements and have them delivered to the user's address.

In some embodiments, a microphone 32 may be present and detect if the fan 16 and or the fan motor 30 is making too much noise. If the noise detected by the microphone 32 exceeds a certain threshold which, may be programmed at the factory or set by user via the user input device 68, the microcontroller 56 may operate the speakers 34 and/or 54 to generate noise canceling sound. The noise canceling sound may make the fan 16 and the fan motor 30 seem quiet to user.

The microphone 32 can also be used as part of a voice command control system programmed in the microcontroller 56. If, for some reason, the lights 20 automatically shut off while the user was still in the bathroom, the user can give the unit a voice command, “Lights.” When the unit 10 receives this command, it will turn the lights 20 back on to the last luminosity percentage or some other set level. The user can use the same voice command of “Lights” to turn the lights off. The user can change the luminosity level with the voice command “Lights [to a spoken percentage].” The user can also control the lights from the unit's wall control.

A humidity sensor 28 will be used to determine the level of humidity in the air. If the humidity level is detected to be greater than a set limit, the unit 10 will automatically turn on its fan 16. The fan 16 will use modulating motor 30 so that it can have a speed range of 0% to 100%. The fan speed is proportionally related to the humidity level. The user can use voice commands such as, “Fan” to turn it on or off or “Fan [to a spoken percentage]” to change the intensity (speed) of the fan 16. The user can also control the fan speed from the unit's wall control.

In some embodiments, an infrared heating element 18 will be energized automatically when the fan 16 is automatically turned on based on humidity. The heating element 18 can also automatically energize based on motion detection sensors 22 (user selectable). The user can also use the voice command, “Heat” to turn the heating elements 18 on or off. The infrared heating element 18 can also be controlled from the unit's wall control 46.

The unit 10 can be paired to multiple users' smart devices via Bluetooth or other communication protocol. Once a smart device 70 is paired to the unit 10, the user can wirelessly play music (or any sound file) through the unit's speakers 34 and/or 54. The unit 10 will use a built-in microphone 32 to automatically adjust the volume based off the ambient noise level of the room. So the louder the ambient noise, the more the unit 10 will increase the volume level. If the ambient noise is reduced, the unit 10 will decrease the volume. The user can also control the volume as well as basic control via voice commands; “Pause,” “Back,” “Next,” “Up,” and “Down.” The user can also adjust the volume from the unit's wall control 46 or second remote device 70. If the user receives a phone call while the unit 10 is connected to their device 70, the unit 10 will dim the music (if playing) and announce to the user who is calling. The user can use voice commands such as “Answer,” “Ignore,” or “End.”

The speakers 34, may play music or other programming which may be input by a user via a wired or wireless connection. For example, a user may be able to use a remote unit 70 such as a smart phone, tablet computer, PC, or any other remote device to send music or other audio programming to the microcontroller 56 which in turn plays that programming via the speakers 34. As discussed above, the speakers 34 can be located at at least one or more locations such as on the ventilation unit 10 or at remote locations which include but are not limited to a wall-mounted remote unit 46.

It should be understood that while the above description describes a user operating the unit 10 using voice command, a user input device 68 may also be used to give the commands. Furthermore, when the above description describes a user operating the device using a user input device 68, it should be understood that a voice activated system may also be used to control the unit 10.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

What is claimed is:

1. A ventilation fan and light system comprising:
a controller;
a sensor operatively connected to the controller;
a fan switch operatively connected to the fan;
a light operatively connected to the controller;
a dimmer operatively connected to the light and the controller to operate the light at various levels of brightness;
a light switch operatively connected to the controller and configured to selectively operate the light; and
a first input device operatively connected to the controller to input to the controller a signal associated with any one of the following: a time of day and a detected level of ambient light.

2. The system of claim 1, further comprising a user input operatively connected to the controller and configured to allow a user to set rules for how much the light is dimmed.

3. The system of claim 1, further comprising an infrared heat lamp operatively connected to the controller.

4. The system of claim 1, wherein the switch comprises a motion sensor.

5. The system of claim 1, further comprising a humidity sensor operatively connected to the controller and the controller is operatively connected to the fan to activate the fan when a level of humidity is detected.

6. The system of claim 1, wherein the first input device comprises a light sensor.

7. The system of claim 1, further comprising an air freshener dispenser operatively connected to the controller.

8. The system of claim 7, further comprising an odor detector operatively connected to the controller and the controller operates the air freshener dispenser based on a signal received from the odor detector.

9. The system of claim 1, further comprising a microphone operatively connected to the controller and the controller is configured to operate the ventilation fan and light by voice command.

10. The system of claim 1, further comprising a wireless communicator operatively connected to the controller, the wireless communicator configured to allow the controller to communicate wirelessly with an external device.

11. The system of claim 10, further comprising speakers operatively connected to the controller.

12. The system of claim 11, wherein the controller and speakers are configured to play audio files received from the external device.

13. The system of claim 11, further comprising a microphone operatively connected to the controller and the controller is configured to monitor a sound associated with a fan motor and the controller is configured to control the speakers to generate a fan noise cancelling sound.

14. The system of claim 10, wherein the controller is configured to receive and respond to commands from a remote device.

15. The system of claim 14, wherein the remote device includes a user input that includes an actuator to activate at least one of the following features of the system; light, heat, fan, speakers and air freshener dispenser.

16. The system of claim 15, wherein the remote device includes at least one of the following system features which is operatively connected to the controller; a motion sensor, a motion sensor, an odor sensor, a humidity sensor, a microphone, a user input device; and a user display.

17. The system of claim 14, wherein the remote device is one of a smart phone, a tablet computer, a remote control, and a personal computer.

18. A method of providing ventilation and light comprising;
connecting a light to a controller;
connecting a fan to the controller;
connecting a dimmer to the light and the controller;
connecting a switch to the controller; and
configuring a signal generator to generate a signal to the controller wherein the signal is associated with one of either a time of day and an amount of ambient light.

19. The method of claim 18, further comprising connecting a heater to the controlled and a user input to the controller wherein the user input is configured to provide instructions to the controller to operate the light, fan, dimmer and heater.

20. A ventilation fan and light system comprising:
a means for controlling fan and light system;
a means for sensing connected to the means for controlling;
a means for switching operatively connected to a means for moving air;
a light operatively connected to the means for controlling;
a means for controlling brightness of the light operatively connected to the means for controlling;
a means for switching operatively connected to the light; and
a means for inputting data to the means for controlling, the means for inputting data configured to input data associated with one of either: a time of day and an amount of light detected by the means for sensing.
